(19) United States
(12) Patent Application Publication
(10) Pub. No.: US 2012/0136195 A1
(43) Pub. Date: May 31, 2012

(54) DRUG DELIVERY SYSTEM OF TARGET DRUG AND METHOD OF IMPLEMENTING THE SAME

(76) Inventors: Yunn-Hwa MA, Kwei-Shan (TW); Hao-Li Liu, Kwei-Shan (TW); Tony Wu, Kwei-Shan (TW); Shao-Kai Liu, Kwei-Shan (TW)

(21) Appl. No.: 13/115,735

(22) Filed: May 25, 2011

(30) Foreign Application Priority Data
Nov. 26, 2010 (TW) 099141081

(51) Int. Cl. A61N 2/10; A61N 7/00

(52) U.S. Cl. 600/12

(57) ABSTRACT

A drug delivery system of a target drug and method of implementing the same. Said drug delivery system of target drug comprising: a plurality of drug-carrying magnetic particles, a magnetic field generation device, and an ultrasound generation device. Said magnetic field generation device generates a magnetic field to guide said magnetic particles to a target activity position; and said ultrasound generation device generates ultrasound to make said magnetic particles into a suspension state, thus increasing reaction area, enhancing local reaction of drug, such that local retention of drug is able to achieve maximum therapeutic effects at reduced drug dosage.

### BACKGROUND OF THE INVENTION

1. Field of the Invention

The present invention relates to a drug delivery system of drug and method of implementing the same, and in particular to a drug delivery system of target drug and method of implementing the same.

2. The Prior Arts

In general, magnetic particle serving as drug carrier has the unique magnetic characteristic that, it can be guided by the attraction of an outside magnetic force applied at a proper distance to stay around the target in achieving therapeutic effect. Therefore, this kind of magnetic particles can be developed to carry drugs in achieving therapeutic effect at reduced dosage.

By way of example, conventional thrombolytic drugs usually has the drawback of narrow therapeutic window and risk of increased hemorrhagic transformation, so that while delivering drug 11 to the blood clot portion 10 in an non-exclusive manner, drug will disperse evenly in blood vessel 13, instead of dispersing only around the blood clot 16 in the vessel, as shown in FIG. 1. Therefore, it could cause some detrimental side effects after therapeutics. For this reason, a better way of delivering drug is to use carrier with targeting function to achieve exclusive target drug delivery. However, as shown in FIG. 2, in magnetically guiding the thrombolytic drugs, when an outside magnetic force is applied on the blood clot portion 10, the drug-carrying magnetic particles 12 tend to gather closely around certain portion of the blood clot, thus reducing its therapeutic efficacy. Therefore, the position of the magnetic field 14 has to be moved constantly around the site of the blood clot to cause suspension of particles in achieving larger reaction surface area. However, in moving the position of the magnetic field, it is liable to cause flowing away and loss of the drug-carrying magnetic particles.

Therefore, at present, the design and performance of the conventional drug delivery system of target drug and method of implementing the same are not quite satisfactory, and it has much room for further improvement.

### SUMMARY OF THE INVENTION

In view of the problems and shortcomings of the prior art, the present invention provides a drug delivery system of target drug and method of implementing the same, so as to overcome the problem and shortcomings of the prior art.

A major objective of the present invention is to provide a drug delivery system of target drug and method of implementing the same, wherein, magnetic field is used to guide the drug-carrying magnetic particles to the target activity position, and in combination with the ultrasound to realize magnetic particles suspension, increase its reaction area, and enhance local reaction of drug, in realizing local retention of drug in a suspension state while retaining its activity and achieving therapeutic efficacy at lower drug dosage.

In order to achieve the above-mentioned objective, the present invention provides a drug delivery system of target drug, comprising: a plurality of drug-carrying magnetic particles, a magnetic field generation device, and an ultrasound generation device. Wherein, the magnetic field generation device generates a magnetic field to guide the magnetic particles to a target site; and the ultrasound generation device is used to make the magnetic particles to be in a suspension state.

Further scope of the applicability of the present invention will become apparent from the detailed description given hereinafter. However, it should be understood that the detailed description and specific examples, while indicating preferred embodiments of the present invention, are given by way of illustration only, since various changes and modifications within the spirit and scope of the present invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The related drawings in connection with the detailed description of the present invention to be made later are described briefly as follows, in which:

FIG. 1 is a schematic diagram of drug delivery through non-exclusive drug delivery according to the prior art;

FIG. 2 is a schematic diagram of when transporting drug-carrying magnetic particles to blood clot site through outside magnetic field to proceed with drug delivery, the drug-carrying magnetic particles stay and concentrate around blood clot according to the prior art;

FIG. 3 is a schematic diagram of a drug delivery system of target drug according to an embodiment of the present invention;

FIG. 4 is a flowchart of the steps of a method of implementing a drug delivery system of target drug according to an embodiment of the present invention;

FIG. 5 is a schematic diagram of transporting drug-carrying magnetic particles to a blood clot site to proceed with drug delivery through utilizing a drug delivery system of target drug according to an embodiment of the present invention;

FIG. 6(a) is a photograph taken in an experiment, wherein ultrasound trigger magnetic particles into a suspension state;

FIG. 6(b) is a dispersion ratio quantization graph for the magnetic particles triggered into suspension by the ultrasound;

FIG. 7(a) is a photograph taken in an experiment, wherein ultrasound trigger magnetic particles into a suspension state;

FIG. 7(b) is a dispersion ratio quantization graph for the magnetic particles triggered into suspension by the ultrasound;

FIG. 8 is a schematic diagram of an experiment design for calculating acoustic power according to the present invention;

FIG. 9 shows impact of various ultrasound acoustic pressure on thrombolytic drugs rtPA, and rtPA fixed on the magnetic particles according to the present invention;

FIG. 10 shows impact of various numbers of ultrasound pulses on thrombolytic drugs rtPA, and rtPA fixed on the magnetic particles according to the present invention; and

FIGS. 11(a) and 11(b) are schematic diagrams of non-focus type probe and focus type probe respectively according to the present invention.

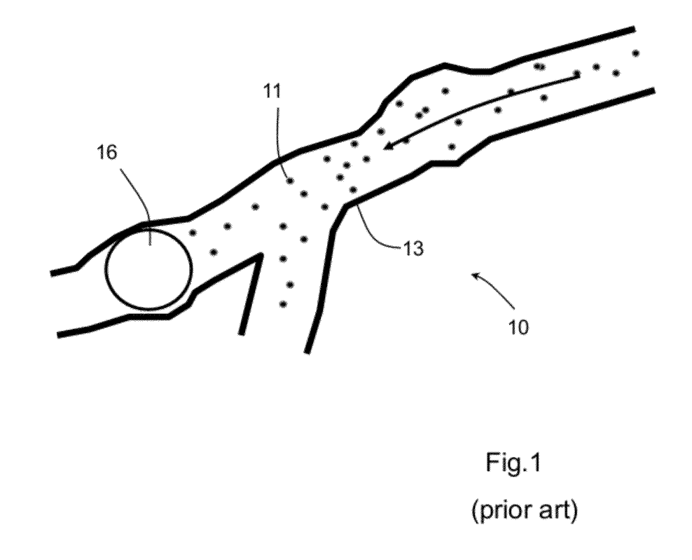

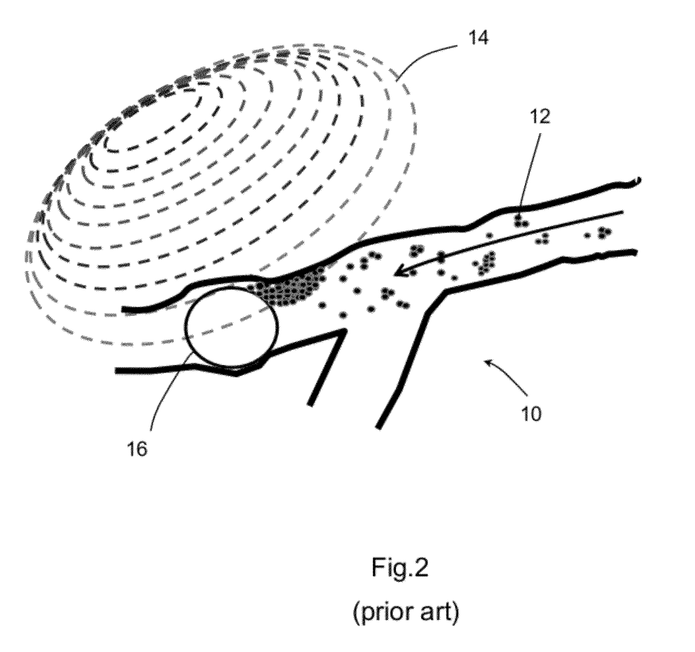

### DETAILED DESCRIPTION OF THE PREFERRED EMBODIMENT

The purpose, construction, features, functions and advantages of the present invention can be appreciated and understood more thoroughly through the following detailed description with reference to the attached drawings.

The purpose of the present invention is to overcome the problems and shortcomings of the conventional target drug delivery therapeutic method that, the drug-carrying magnetic particles tend to stay and concentrate in a local portion due to the outside applied magnetic field, thus leading to reduction of therapeutic efficacy. Therefore, the present invention provides a drug delivery system of target drug and a method of implementing the same, so as to realize suspension of drug-carrying magnetic particles, increase its reaction area, and enhance local reaction of drug, in causing local retention of drug in a suspension state while keeping its activity, hereby achieving therapeutic effect at lower drug dosage, and reduction of side effects of drugs.

The present invention may be used in intravascular application (including artificial vessels), such as thrombolysis application to deliver thromolytic agents (plasminogen activators). The present invention may be used to induce vasodilation (direct vasodilators), protect endothelial function (anti-oxidants, statins etc), and inhibit vascular smooth muscle proliferation/migration (growth factor inhibitors, receptor tyrosine kinase inhibitors etc). The term “drug” means chemicals, proteins, nucleotides etc.

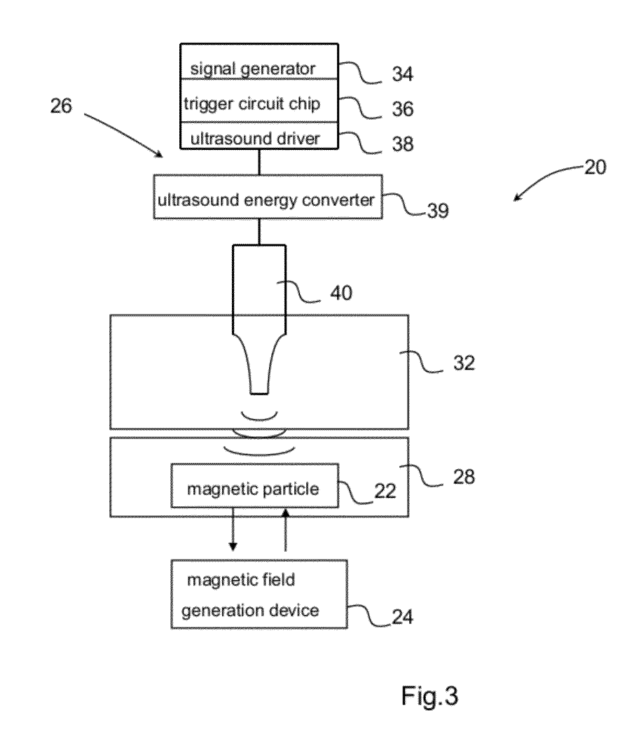

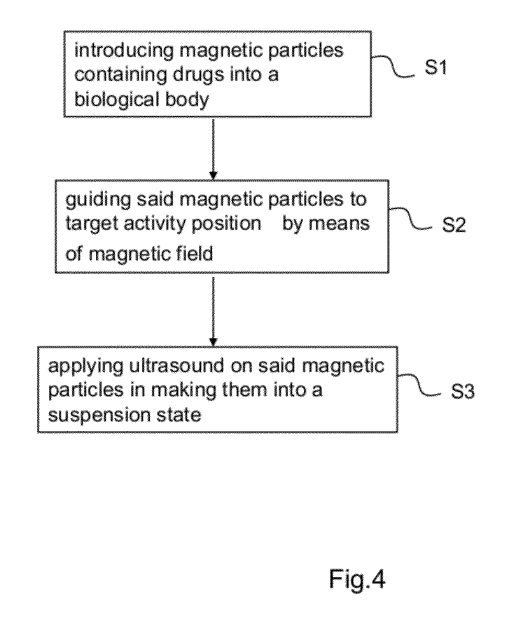

Refer to FIGS. 3,4, and 5 respectively at the same time for a schematic diagram of a drug delivery system of target drug according to an embodiment of the present invention; a flowchart of the steps of a method of implementing a drug delivery system of target drug according to an embodiment of the present invention; and a schematic diagram of transporting drug-carrying magnetic particles to a blood clot site to proceed with drug delivery through utilizing a drug delivery system of target drug according to an embodiment of the present invention. In the present embodiment, a thrombolytic drug is taken as an example, however, the present invention is not limited to the therapeutics of blood clot in a blood vessel.

Firstly, as shown in FIG. 3, the drug delivery system 20 of target drug comprises a plurality of drug-carrying magnetic particles 22, such as iron oxide particles; a magnetic field generation device 24; and an ultrasound generation device 26.

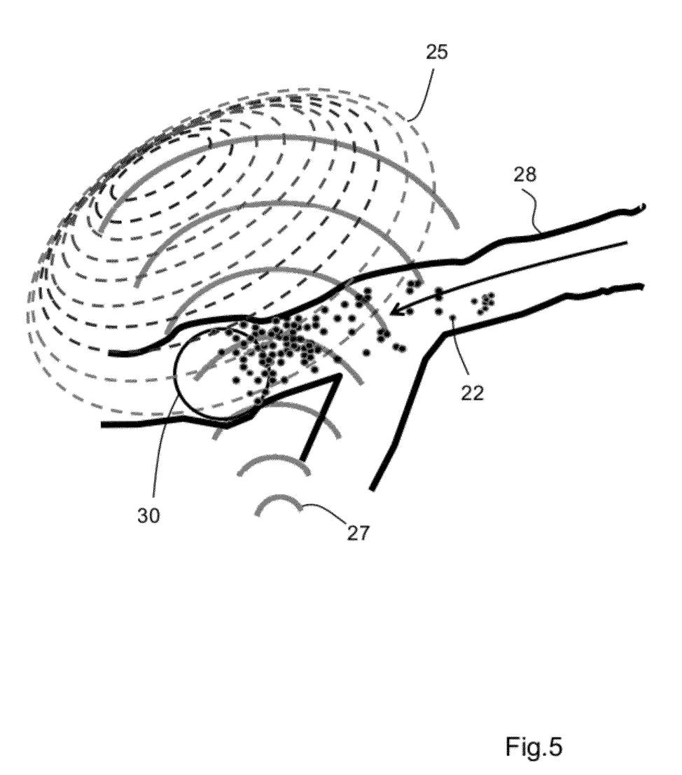

In operation, firstly, as shown in step S1, introducing the magnetic particles 22 containing drug into a biology body, such as a blood vessel 28, wherein, the drug can be selected from thrombolytic drugs, such as, rtPA, urokinasec, or streptokinase; next, as shown in step S2, disposing a magnetic field generation device 24 outside the biology body, for generating a magnetic field 25 to guide the magnetic particles 22 to a target activity position, for example a blood clot 30 as shown in FIG. 5; then, as shown in step S3, disposing also an ultrasound generation device 26 outside the biology body to generate ultrasound 27, which is applied onto the magnetic particles 22 through a medium 32, so as to cause the magnetic particles 22 into a suspension state around the target activity position, hereby increasing its reaction area, as shown in FIG. 5. By the way, the steps S2 and S3 mentioned above can be performed simultaneously.

In the present embodiment, the frequency of ultrasound generated by the ultrasound generation device 26 can be in a range of 20 kHz to 50 MHz.

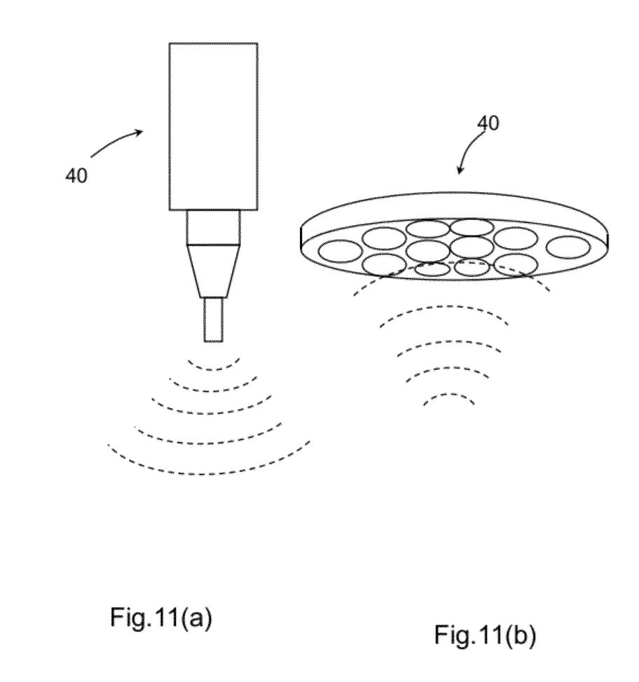

In addition, as shown in FIG. 3, the ultrasound generation device 26 mentioned above may include: a signal generator 34, for generating a sine wave cluster signal; a trigger circuit single chip 36, which receives the sine wave cluster signal to generate a trigger circuit signal, for controlling ultrasound parameters, such as varying pulse width and pulse repetition frequency (PRF); an ultrasound driver 38, which receives the trigger circuit signal to drive an ultrasound energy converter 39 into generating an ultrasound; and a probe 40, which receives the ultrasound and emits focused ultrasound or non-focused ultrasound. The probe 40 just mentioned can be of a non-focused type or a focused type as shown in FIGS. 11(a) and 11(b) respectively.

In the system configuration of the present embodiment, the ultrasound mentioned above can be transmitted to the biology body through a medium 32, such as, water or ultrasound conduction gel.

The magnetic field generation device 24 mentioned above can be a permanent magnet, such as a natural magnet, an Nd—Fe—B (Nd₂F₁₄B) magnet, an Sm—Co (Sm—Co) magnet, or an Al—Ni—Co (AlNiCo) magnet; or a non-permanent magnet, such as an electromagnet, or a superconducting magnet.

In the following, an experiment is described in verifying the efficacy of the present invention.

The instrument utilized in the experiment is a 28 kHz ultrasound system available on the market (K-Sonic Inc., Taipei, Taiwan), and after modification, it adopts a single trigger circuit to control ultrasound parameters, including pulse length, and pulse repetition frequency (PRF). The experiment instrument for the particle suspension image processing includes: a water bath; an ultrasound probe fixing frame; a permanent magnet; a blood vessel simulation tube; a microscope; an object lens modified CCD camera for taking images; a goose neck lamp installed to raise illumination of image, such that the image is transmitted back to a computer for further processing; and a ultrasound probe, that is triggered through utilizing degassed water or ultrasound conduction gel as medium. The experiment evaluation is conducted by means of a blood vessel simulation system set up through simulating an in vivo flowing structure, and a microscope is utilized to observe the magnetic particles injected into the test tube of simulated blood vessel model, (the magnetic particles are attached and fixed onto the simulated tube by means of a 0.44TNdFeB magnet), the impact on magnetic particles under an outside applied magnetic field, and on the magnetic particles in the blood vessel simulation tube triggered by the ultrasound, hereby obtaining better parameters for concussing and dispersing the magnetic particles into a suspension state. In order to observe the increased dispersion of magnetic particles under ultrasound, and the capability of maintaining dispersion under various different parameters, the adjusting parameters utilized may include duty cycle and pulse repetition frequency (PRF). As such, a CCD camera is utilized to take pictures of magnetic particle dispersions during each time interval of applying ultrasound by utilizing a Micrometric SE image software; subsequently, perform post treatment of pictures taken, dispersion ratio calculation, and area analyses by using a software Image J, and after making necessary modifications and improvements, the evaluations of magnetic particle suspensions and dispersions for various ultrasound parameters can be compared to make the adjustments required.

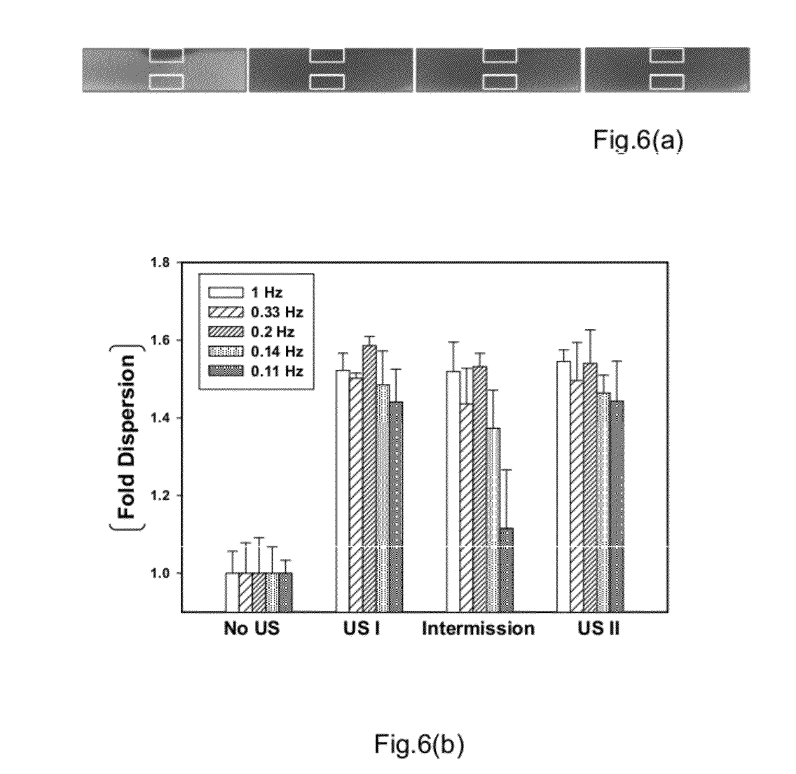

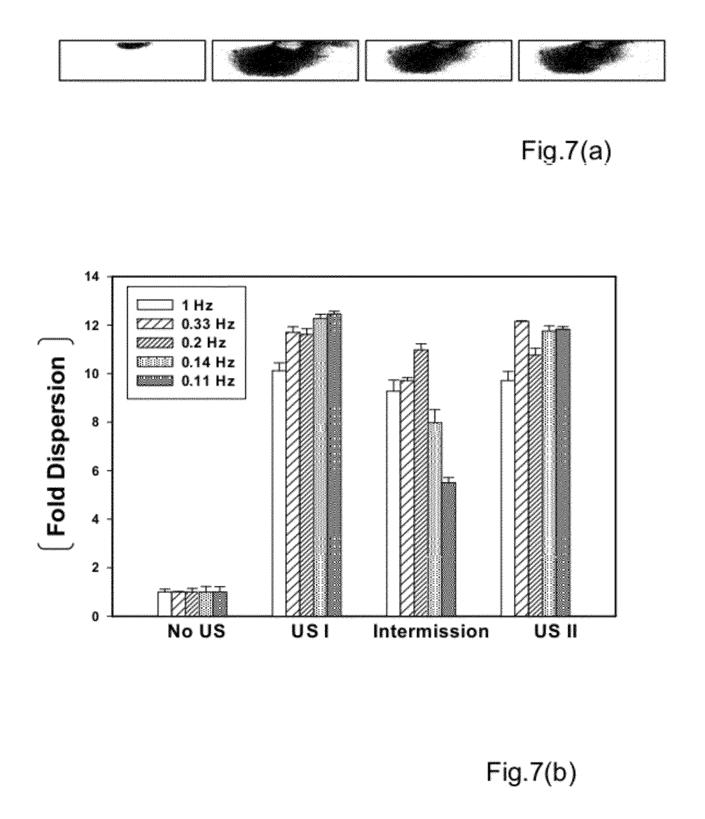

Refer to FIG. 6(a) for a photograph taken in an experiment, wherein ultrasound trigger magnetic particles into a suspension state. As shown in FIG. 6(a), program analysis is used to fetch the magnetic field in upper portion that make the magnetic particles to concentrate, in contrast to the more clearer lower portion. Moreover, as shown in FIG. 6(b), dispersion ratio is used to calculate and compare the impacts of various ultrasound parameters on the magnetic particle suspension, under the conditions of not applying ultrasound, applying the first ultrasound, intermission, applying the second ultrasound. The definition of dispersion ratio is used to fix the region of the image with upper and bottom areas, and calculate the ratio of the gray scale value. Then refer to FIG. 7(a) for a photograph taken in an experiment, wherein ultrasound trigger magnetic particles into a suspension state. As shown in FIG. 7(a), gray level values are used for analysis, such that area analysis quantization is performed through using fixed range of threshold values, as shown in FIG. 7(b). As such, through the two sets of diagrams mentioned above, the feasibility of ultrasound triggering the magnetic particles into suspension can be verified.

The ultrasound energy evaluation can be performed on acoustic power and negative peak pressure simultaneously for calculations and corrections. The total acoustic power of continuous mode is measured at around 30 W. The acoustic power is measured and calculated by using a calibrated ultrasound power meter, and the peak pressure distribution is measured and calculated by a needle hydrophone under various conditions. The acoustic power calculation experiment is conducted through using the acoustic pressure produced by ultrasound and based on various distances to the probe and various mediums, that includes the silicon tube used in blood vessel simulation system, the aorta blood vessel taken from Sprague Dawley mouse for comparison. The reason for selecting this blood vessel wall is that, the size of the blood vessel attached window has to be close to that of the hydrophone probe, and this experiment is used to evaluate the acoustic pressure of the blood vessel simulation system, and the real blood vessel penetrated by ultrasound, with MPa as the measurement unit. From this data, acoustic pressure data under various parameters can be obtained.

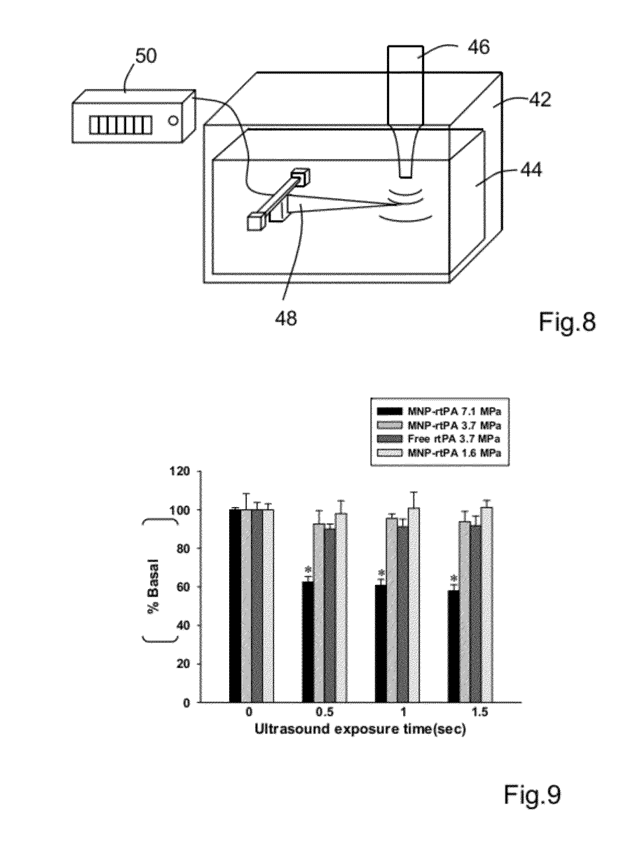

Refer to FIG. 8 for a schematic diagram of an experiment design for calculating ultrasound acoustic pressure according to the present invention. As shown in FIG. 8, wherein, degassed water 44 is filled into water bath 42, probe 46 of ultrasound system is fixed onto an adjustable fixer (not shown), and a needle hydrophone 48 is fixed in the water and is connected to an oscilloscope 50 for signal analysis. In order to get the entire wave, sample mode is used to measure the peak-to-peak voltage, and convert it to acoustic pressure with smaller variations, as such, through experiments each of different distance and different medium material, the ultrasound pressure under various parameters can be analyzed to make adjustments required.

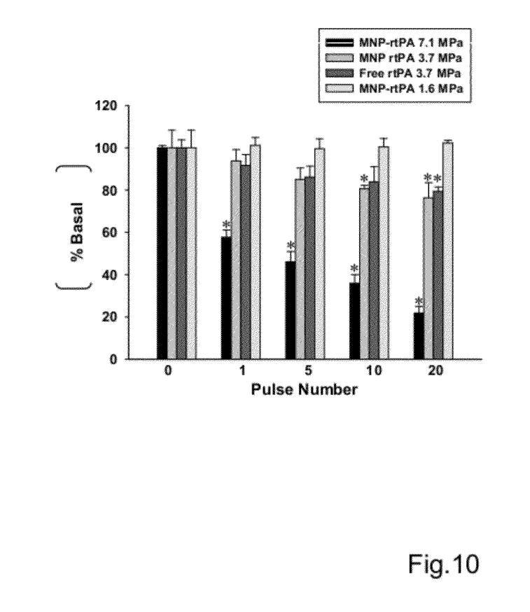

In the evaluation of ultrasound power and parameter adjustment, and their impacts on enzyme activity, in order to ascertain whether ultrasound could affect drug activity, thus evaluating correctly the benefit of the present invention, therefore in an experiment, various acoustic pressure are given, and evaluations are made for activity of drug on the magnetic particles after applying various numbers of pulses. Refer to FIG. 9, which shows impacts of various ultrasound acoustic pressure on thrombolytic drugs rtPA (recombinant tissue plasminogen activator), and on rtPA fixed on the magnetic particles according to the present invention. From the results it can be known that, at acoustic pressure of 7.1 MPa, it could cause apparent loss of enzyme activity, while at lower acoustic pressure, the enzyme activity will not be affected. Also, refer to FIG. 10, which shows impacts of various numbers of ultrasound pulses on thrombolytic drugs rtPA, and rtPA fixed on the magnetic particles according to the present invention. As shown in FIG. 10, in this type of experiment, ultrasound triggers are given 1, 5, 10, and 20 times, to perform analysis about their impacts on rtPA, and rtPA fixed on the magnetic particles, thus obtaining data of impacts of various acoustic pressure on enzyme activity after applying successive ultrasound. From the result it can be confirmed that, the present invention is able to cause suspension of magnetic particles, while reducing as much as possible its impact on the drug carried by the magnetic particles.

Summing up the above, the present invention provides a drug delivery system of target drug and method of implementing the same, which combines ultrasound, magnetic field, and magnetic particles to perform therapeutics of drug target, raising the safety and efficacy of drug application, and achieving maximum therapeutic effects at reduced drug dosage.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A drug delivery system of target drug, comprising:
a plurality of magnetic particles, used as drug carriers;
a magnetic field generation device, used to generate a magnetic field and guide said magnetic particles to a target activity position; and
an ultrasound generation device, used to generate ultrasound, so as to make said magnetic particles into a suspension state.

2. The drug delivery system of target drug as claimed in claim 1, wherein said magnetic particle is a ferromagnetic particle.

3. The drug delivery system of target drug as claimed in claim 1, wherein said ultrasound generation device applies ultrasound on said target activity position through a medium, said medium is aqueous solution or ultrasound conduction gel.

4. The drug delivery system of target drug as claimed in claim 1, wherein said ultrasound generation device comprises:
a signal generator, used to generate a sine wave cluster signal;
a trigger circuit single chip, used to receive said sine wave cluster signal, and generate a trigger circuit signal;
an ultrasound driver, used to receive said trigger circuit signal, and drive an ultrasound energy converter to generate an ultrasound; and
a probe, used to receive said ultrasound, and emit focused type or non-focused type ultrasound.

5. The drug delivery system of target drug as claimed in claim 1, wherein
said magnetic particles serve as drug-carriers of a thrombolytic drug.

6. The drug delivery system of target drug as claimed in claim 5, wherein
said thrombolytic drugs is rtPA, urokinasec, streptokinase or plasminogen activators.

7. The drug delivery system of target drug as claimed in claim 1, wherein
said magnetic field generation device is a permanent magnet, selected from a natural magnet, an Nd—Fe—B (Nd₂F₁₄B) magnet, an Sm—Co (Sm—Co) magnet, or an Al—Ni—Co (AlNiCo) magnet.

8. The drug delivery system of target drug as claimed in claim 1, wherein
said magnetic field generation device is an electromagnet, or a superconducting magnet.

9. The drug delivery system of target drug as claimed in claim 1, wherein
frequency of said ultrasound is 20 kHz to 50 MHz.

10. The drug delivery system of target drug as claimed in claim 1, which is used to deliver a drug to induce vasodilation, protect endothelial function, or inhibit vascular smooth muscle proliferation/migration.

11. The drug delivery system of target drug as claimed in claim 1, which is used to deliver a drug in an artificial vessel.

12. A method for implementing a drug delivery system of target drug as claimed in claim 1, comprising following steps:
(a) introducing magnetic particles containing drugs into a biological body;
(b) disposing a magnetic field generation device outside said biological body, guiding said magnetic particles by means of a magnetic field to a target activity position in said biological body; and
(c) disposing an ultrasound generation device outside said biological body, for applying ultrasound on said magnetic particles in making them into a suspension state.

13. The method for implementing drug delivery system of target drug as claimed in claim 12, wherein
said step (b) and said step (c) are performed simultaneously.
